(19) United States
(12) Patent Application Publication
(10) Pub. No.: US 2011/0307974 A1
(43) Pub. Date: Dec. 15, 2011

(54) PLANT GROWTH PROMOTING PROTEIN COMPLEX

(76) Inventors: Gerrit Beemster, Zaamslag (NL); Geert De Jaeger, Evergem (BE); Nubia B. Eloy, Gent (BE); Paulo C.G. Ferreira, Rio de Janeiro (BR); Dirk G. Inze, Moorsel-Aalst (BE); Adriana Hemerly, Rio de Janeiro (BR); Jelle Van Leene, Gent (BE)

(21) Appl. No.: 12/998,832

(22) PCT Filed: Dec. 4, 2009
(86) PCT No.: PCT/EP2009/066419

§ 371 Date: Aug. 22, 2011

(30) Foreign Application Priority Data
Dec. 5, 2008 (EP) 08170792.9

(51) Int. Cl. A01H 5/00; A01H 1/06

(52) U.S. Cl. 800/285; 800/290; 800/298; 800/320; 800/320.1; 800/320.2; 800/320.3

(57) ABSTRACT

The present invention relates to a plant growth promoting protein complex. More specifically, the invention relates to the use of specific proteins from the Anaphase Promoting Complex/Cyclosome for increasing shoot growth rates and/or enhancing cell division rates.

### CROSS-REFERENCE TO RELATED APPLICATIONS

This is a national phase entry under 35 U.S.C. §371 of International Patent Application PCT/EP2009/066419, filed Dec. 4, 2009, published in English as International Patent Publication WO 2010/063833 A2 on Jun. 10, 2010, which claims the benefit under Article 8 of the Patent Cooperation Treaty to European Patent Application Serial No. 08170792.9, filed Dec. 5, 2008.

### TECHNICAL FIELD

The present invention relates to a plant growth promoting protein complex. More specifically, the invention relates to the use of specific proteins from the Anaphase Promoting Complex/Cyclosome for increasing shoot growth rates and/or enhancing cell division rates.

### BACKGROUND

Ubiquitination-mediated proteolysis is a primary mechanism by which the levels of regulatory proteins are controlled. The process of ubiquitination of a substrate involves the activity of a cascade of three enzymes, the ubiquitin-activating enzyme (E1), the ubiquitin-conjugating enzyme (E2), and the ubiquitin-protein ligase (E3). The substrate specificity and regulation of ubiquitination are conferred by the E3 ubiquitin protein ligase, which binds directly to the target protein and is the rate-limiting step in the ubiquitination cascade (reviewed in Hershko and Ciechanover, 1998; and Peters, 2002).

Two structurally related multiprotein E3 ligases, the anaphase-promoting complex/cyclosome (APC/C) and the Skp1/Cullin/F-box protein (SCF) complex drive progression through the eukaryotic cell cycle. The activity of SCF ligases mainly controls the transition from G1/S and G2/M, while APC/C is primarily required for mitotic progression and exit (Morgan, 1999).

APC is one of the most complex molecular machines known to catalyze ubiquitination reactions, as it contains more than a dozen subunits (Yoon et al., 2002; Peters et al., 1996). This complexity is unexpected because many other ubiquitin ligases are only composed of one or a few subunits, meaning that ubiquitin ligase activity does not inevitably depend on multiple subunits. Therefore, it remains puzzling why the APC is composed of so many protein components and what their individual functions are.

APC10 is a subunit of APC/C that contains a Doc 1 (Destruction of Cyclin) domain, which is also found in several other proteins of the ubiquitin-proteasome system. Mutants of APC10 in yeast are known to prevent substrate binding to APC/C^{Cdh1}, suggesting that this subunit may play a role in substrate recognition. Passmore et al. (2003) have demonstrated that APC10 contributes to APC substrate recognition independently of coactivator and it implicates that APC10 acts as a potential APC regulatory subunit.

Biochemical analysis of budding-yeast APC shows that APC10/DOC1 increases the processivity of substrate ubiquitination by enhancing the affinity of the APC-substrate complex (Carrol et al., 2005). Importantly, the interaction between. APC and the activators CDH1 and CDC20 is not affected by loss of APC10/DOC1 function, suggesting that APC10/DOC1 promotes substrate binding directly or in concert with other core APC subunits (Au et al., 2002).

The identification of the complete set of genes encoding the APC subunits in Arabidopsis reinforces the evidence that the basic processes controlled by ubiquitin-mediated proteolysis in plants are similar to other eukaryotes (Eloy et al., 2006). However, the results on gene structure and expression unraveled unique characteristics of the plant APC and it indicates the prospect of flexible complexes that may be particularly required for growth responses needed to adapt to changing environmental conditions (Eloy et al., 2006).

### SUMMARY OF THE INVENTION

Surprisingly, we found that lines overexpressing the APC10 subunit, as well as lines with a loss of function of a Novel Interactor of the APC 10 subunit (SAMBA), showed an increased growth.

A first aspect of the invention is the use of APC10, or a variant thereof, to increase plant growth and/or yield. The use, as indicated here, is the use of the protein and/or the use of a nucleic acid encoding this protein, or the complement thereof. It is including, but not limited to, genomic DNA, cDNA, messenger RNA (including the 5′ and 3′ untranslated regions) and RNAi. “Variants,” as used herein include, but are not limited to, homologues, orthologues and paralogues of SEQ ID NO:1 (APC10 protein) of the incorporated herein Sequence Listing. Homologues of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Orthologues and paralogues encompass evolutionary concepts used to describe the ancestral relationships of genes. Paralogues are genes within the same species that have originated through duplication of an ancestral gene; orthologues are genes from different organisms that have originated through speciation and are also derived from a common ancestral gene. Preferably, the homologue, orthologue or paralogue has a sequence identity at a protein level of at least 50%, 51%, 52%, 53%, 54% or 55%, 56%, 57%, 58%, 59%, preferably 60%, 61%, 62%, 63%, 64%, 65%, 66%, 67%, 68%, 69%, more preferably 70%, 71%, 72%, 73%, 74%, 75%, 76%, 77%, 78%, 79%, even more preferably 80%, 81%, 82%, 83%, 84%, 85%, 86%, 87%, 88%, 89%, and most preferably 90%, 91%, 92%, 93%, 94%, 95%, 96%, 97%, 98%, 99%, or more as measured in a BLASTp (Altschul et al., 1997; Altschul et al., 2005). As a non-limiting example, orthologues of SEQ ID NO:1 are Pt796785 (ₚoplar), Vv00024912001 (vitis), AC187383 (maize) and Os05g50360 (Rice). Increase of plant growth and/or yield is measured by comparing the test plant comprising a gene used according to the invention with the parental, non-transformed plant, grown under the same conditions as control. Preferably, increase of growth is measured as an increase of biomass production. “Yield” refers to a situation where only a part of the plant, preferably an economical important part of the plant, such as the leaves, roots or seeds, is increased in biomass. The term “increase” as used herein means at least a 5%, 6%, 7%, 8%, 9% or 10%, preferably at least a 15% or 20%, more preferably 25%, 30%, 35% or 40% more yield and/or growth in comparison to control plants as defined herein. “Increase of plant growth,” as used herein, is preferably measured as increase of any one or more of leaf biomass, root biomass and seed biomass.

Another aspect of the invention is the use of an APC10 interacting protein, or a variant thereof, or the use of nucleic acid encoding this protein, or the complement thereof to increase plant growth. Indeed, as APC10 is part of a protein complex, its function can be compensated by over- or underexpression of other proteins in the complex. Preferably, the APC10 interacting protein is selected from the list consisting of any one or more of AT2G39090, AT2G20000, AT5G05560, AT3G48150, AT1G06590, AT1G78770, AT4G21530, AT2G04660, AT1G32310, AT2G42260, AT4GA19210, AT3G57860, AT3G16320, AT4G25550, AT5G13840, AT3G48750, AT3G56150 and AT2G06210, or a variant thereof. Even more preferably, the APC10 interacting protein is SAMBA (SEQ ID NO:2), or a variant thereof. “Variants,” as used herein, include, but are not limited to, homologues, orthologues or paralogues of SEQ ID NO:2 (SAMBA protein). Homologues of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. Preferably, the homologue, orthologue or paralogue has a sequence identity at a protein level of at least 40%, 41%, 42%, 43%, 44%, 45%, 46%, 47%, 48%, 49%, preferably 50%, 51%, 52%, 53%, 54% or 55%, 56%, 57%, 58%, 59%, preferably 60%, 61%, 62%, 63%, 64%, 65%, 66%, 67%, 68%, 69%, more preferably 70%, 71%, 72%, 73%, 74%, 75%, 76%, 77%, 78%, 79%, even more preferably 80%, 81%, 82%, 83%, 84%, 85%, 86%, 87%, 88%, 89%, and most preferably 90% 91%, 92%, 93%, 94%, 95%, 96%, 97%, 98%, 99% or more as measured in a BLASTp (Altschul et al., 1997; Altschul et al., 2005). Preferably, the homologue, orthologue or paralogue comprises one or more of the following conserved motifs K(D/E)EA (SEQ ID NO:22) and/or PRS(R/H/C)I (SEQ ID NO:23), even more preferably, the motifs (R/S)K(D/E)EA(M/L/V) and/or F(E/Q/D/G/A)(G/A)PRS(R/H/C)I, most preferably the motive K(D/E)EAXXXLXXXXMXXLXXXVXXLXXXXWXFXXPRSXI (SEQ ID NO:26), where X can be any amino acid. The conserved motifs are shown in FIG. 15. Preferably, the homologue, orthologue or paralogue is a plant protein, even more preferably, a plant protein with the percentage identity and the conserved motif. Preferably, the homologue, orthologue or paralogue is biologically active, as measured by its interaction with APC10, in vitro or in vivo. As a non-limiting example, orthologues of SAMBA (SEQ ID NO:2) are selected from the list consisting of SEQ ID NO:3-SEQ ID NO:21.

In one preferred embodiment, APC10 is overexpressed. In another preferred embodiment, the expression of SAMBA is repressed or completely eliminated. Overexpression or repression refers to the expression in the modified plant, compared with the non modified parental plant, grown under the same conditions. Methods for overexpressing genes or repressing gene expression are known to the person skilled in the art. Overexpression can be realized by, as a non-limiting example, placing the coding sequence of the gene under control of a strong promoter, such as, but not limited to, the CMV 35 S promoter. Alternatively, overexpression can be realized by increasing the copy number of the gene. Repression of gene expression can be realized, as a non-limiting example, by gene silencing, antisense RNA or by RNAi. Design of RNAi is known to the person skilled in the art. As a non limiting example, RNAi can be designed with Web micro RNA designer (Ossowki et al., 2005-2009). RNAi can be directed against a part of the 5′ untranslated terminal region, against a part of the coding sequence, and/or against the 3′ terminal region of the mRNA. Some non-limiting examples of target sequences are listed in Table 1.

Therefore, another aspect of the invention is the use of RNAi against a nucleic acid encoding SAMBA or a variant thereof, as defined above, to increase plant growth. RNAi will target only a part of the nucleic acid, whereby the target sequence can be situated in the coding sequence, or in the 5′ or 3′ untranslated regions of the nucleic acid encoding SAMBA or variant.

Overexpression or repression of expression of a target gene can be obtained by transfer of a genetic construct, intended for overexpression or repression of expression into a plant. The transfer of foreign genes into the genome of a plant is called transformation. Transformation of plant species is a fairly routine technique known to the person skilled in the art. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include, but are not limited to, agrobacterium-mediated transformation, the use of liposomes, electroporation, chemicals that increase free DNA uptake, injection of the DNA directly into the plant, particle gun bombardment, transformation using viruses or pollen and microprojection.

Preferably, the plant as used for this invention is selected from the group consisting of Arabidopsis thaliana, Brassicus sp., Glycine max, Medicago truncatula, Vitis vinifera, Populus sp., Solanum sp., Beta vulgaris, Gossypium hirsutum, Avena sativa, Hordeum vulgare, Triticum aestivum, Oryza sativa, Phyllostachys edulis, Miscanthus sp., Panicum virgatum, Zea mays, Saccharum officinarum, Sorghum bicolor and Ricinus communis. In a preferred embodiment, the plant is a crop plant, preferably a monocot or a cereal, even more preferably it is a cereal selected from the group consisting of rice, maize, wheat, barley, millet, rye, sorghum and oats.

Still another aspect of the invention is a transgenic plant, comprising a RNAi against a nucleic acid encoding SAMBA (SEQ ID NO:2) or a variant thereof. A “transgenic plant” as used herein is a plant, comprising a recombinant DNA construct, whereby the recombinant DNA construct might be introduced directly by transformation, or indirectly by inbreeding. RNAi against a nucleic acid against SAMBA means that the RNAi is capable of down-regulating the wild-type expression of SAMBA. Preferably, the transgenic plant is selected from the group consisting of Arabidopsis thaliana, Brassicus sp., Glycine max, Medicago truncatula, Vitis vinifera, Populus sp., Solanum sp., Beta vulgaris, Gossypium hirsutum, Avena sativa, Hordeum vulgare, Triticum aestivum, Oryza sativa, Phyllostachys edulis, Miscanthus sp., Panicum virgatum, Zea mays, Saccharum officinarum, Sorghum bicolor and Ricinus communis. More preferably, the transgenic plant is a crop plant, preferably a monocot or a cereal, even more preferably it is a cereal selected from the group consisting of rice, maize, wheat, barley, millet, rye, sorghum and oats.

### BRIEF DESCRIPTION OF THE FIGURES

FIG. 1: APC10 expression. Q-PCR analyses of APC10 expression in total seedlings of three-week-old plants.

FIG. 2: Phenotypic analysis of APC10^{OE} lines. Two-week-old plants in vitro grown wild-type (left panel) and APC10^{OE} plants (right panel).

FIG. 3: Kinematic Analysis of Leaf Growth of the First Leaf Pair of Wild-Type (Col-0) and APC10 Overproducing Plants. (Panel A) Leaf blade area. (Panel B) Epidermal cell number on the abaxial side of the leaf. (Panel C) Epidermal cell size on the abaxial side of the leaf.

FIG. 4: Leaf Measurement of three-week-old soil-grown wild-type Columbia and APC10^{OE} plants. FIG. 4A—Leaf area and leaf length line 5.3, FIG. 4B—Leaf area and leaf length line 2.3. The leaf area and leaf length of the wild-type is indicated by the yellow line.

FIG. 5: Fresh and Dry weight measurements of three-week old plants. Panel A—Fresh weight of shoot in APC10^{OE} and WT plants 22 days old. Panel B—Dry weight of shoot in APC10^{OE} and WT plants 22 days old.

FIG. 6: Ploidy level distribution of the first leaves: Panel A—days 14 and Panel B—18. Panel C—wild-type, APC10^{OE}5.3 and APC10^{OE}2.3 plants were measured by flow cytometry.

FIG. 7: Molecular analysis of SAMBA Knockout plants. Panel A—Schematic representation of exon (boxes) and intron (lines) structure of SAMBA. White triangles indicate T-DNA insertion sites. Panel B—SAMBA expression. Q-PCR analyses of SAMBA expression in two first leaves of two-week-old plants.

FIG. 8: Phenotypic analysis of SAMBA knockout lines. Two-week-old in vitro grown SAMBA knockout (left panel) and wild-type plants (right panel). Panel A—SAMBA Knockout (SALK_{—}018488) and wild-type plants. Panel B—SAMBA Knockout (SALK_{—}048833) and wild-type plants.

FIG. 9: Leaf Measurement of three-week-old plants grown in vitro and in vivo. Panel A—Leaf series measurement from 22-day-old plants grown in vitro Columbia (line) and SAMBA knockout plants (blocks). Panel B—Representative picture from the measurement of Panel A. Panel C—Leaf series measurement from 22-day-old plants grown in vivo Columbia (light line) and SAMBA knockout plants (dark line).

FIG. 10: Fresh and dry weight measurement of three-week-old plants. Panel A—Shoot fresh weight of SAMBA and wild-type control plants. Panel B—Shoot dry weight of SAMBA and wild-type control plants.

FIG. 11: Leaf 1 and 2 measurement of 12- and 15-day-old plants of wild-type and SAMBA Knockout plants and Ploidy-level distribution of the first leaves of 14-day-old wild-type and SAMBA Knockout plants. Black rectangle (wild-type) and Grey rectangle (SAMBA Knockout) (Panel A) Leaf blade area (mm²); (Panel B) Epidermal cell number on the abaxial side of the leaf; (Panel C) Ploidy level (%) of wild-type and SAMBA Knockout plants.

FIG. 12: Root measurement of two-week-old plants. Panel A—Primary root measurement of wild-type and SAMBA Knockout plants. Panel B—Representative picture from the measurement of Panel A. Panel C—Root fresh weight measurement. Panel D—Root dry weight measurement.

FIG. 13: Seed size measurement of wild-type and SAMBA Knockout plants.

FIG. 14: Mannitol experiment. Wild-type and SAMBA Knockout plants grown under 25 mM of Mannitol condition and control experiment plants were grown without Mannitol.

FIG. 15: alignment of SAMBA variants, showing the conserved motifs. Arath: Arabidopsis thaliana; Brana: Brassicus napus; Glyma: Glycine max; Medtr: Medicago truncatula; Vitvi: Vitis vinifera; Poptr: Populus tremula; Solly: Solanum lycopersicon; Betvu: Beta vulgaris; Avesa: Avena sativa; Horvu: Hordeum vulgare; Triae: Triticum aestivum; Orysa: Oryza sativa; Phyed: Phyllostachys edulis; Panvi: Panicum virgatum; Zeama: Zea mays; Sacof: Saccharum officinarum; Sorbi: Sorghum bicolor.

### DETAILED DESCRIPTION OF THE INVENTION

### EXAMPLES

### Materials and Methods to the Examples

### Cloning

Cloning of transgenes encoding tag fusions under control of the constitutive Cauliflower tobacco mosaic virus 35S promoter, transformation of Arabidopsis cell suspension cultures, protein extract preparation, TAP purification, protein precipitation and separation were done as described (Van Leene et al., 2007 and 2008).

The genome version of Arabidopsis thaliana (www.arabidopsis.org) was searched for homolog of the APC10 gene using a BLAST program. A sequence of 579 bp and approximately 21 KDa was identified in the TAIR database. The coding region of APC10 (AT2G18290) was used to design specific primers (Attb1APC10 ggggacaagtttgtacaaaaaagcaggcttcacaatggcgacagagtcatcggaat (SEQ ID NO:27) and Attb2APC 10 ggggaccactttgtacaagaaagctgggtatgttcttcaaacttctcctgctc (SEQ ID NO:28)) to isolate the respective cDNA and it was amplified directly by PCR from tissues of Arabidopsis thaliana ecotype Columbia.

The PCR reaction was performed using the Pfx Kit (Invitrogen) according to the manufacturer's instructions. The PCR fragment, referring to complete cDNA from APC10 gene was introduced into pDONr 201 using the Gateway system (Invitrogen) by attBXattP recombination sites and subsequently recombined into the pK7WG2 vector by attL XattR sites recombination. The sequence was confirmed by sequencing.

The APC10_pK7WG2 construction was used to transform Arabidopsis thaliana by the flower-dip method (Clough and Bent, 1998).

### Plant Material

SAMBA knockout plants (seed code: SALK_{—}048833 and SALK_{—}018488) were obtained from the Salk collection (WorldWideWeb//signal.salk.edu/). Twenty plant genotypes of each line were determined by PCR with specific primers for T-DNA insertion element and for SAMBA (LP_atgacgaaacaccgaaaacac (SEQ ID NO:29) and; RP_agttttatggtcggtcacacg (SEQ ID NO:30) for Salk 018488 and LP_ccattgggatcattactgctg (SEQ ID NO:31); RP_aaaggaaacgtgacgattgtg (SEQ ID NO:32) for Salk 048833 and LBb1_{—}3 attttgccgatttcggaac (SEQ ID NO:33) for the left T-DNA border primer).

Among 20 plants, we found two individual homozygous of each line. The presence of T-DNA insertion and absence of the wild-type gene was confirmed by genomic PCR from leaves of 15 days old plants. These plants were selected to produce more seeds and for subsequent analysis. Q-PCR using specific primers (SAMBA_Fwd gctggtctagacgatttcca and SAMBA_Rev-gcttcacttcacctcctttc) for SAMBA was performed to confirm the absence of mRNA of SAMBA.

Arabidopsis plants (ecotype Col-0) were transformed with the APC10_pK7WG2 construction by the floral dip method.⁽¹⁰⁾

Transgenic lines (APC10^{OE}) were identified by selection in 50 mg/l kanamycin in germination medium and later transferred to soil for optimal seed production, and selection of T3 homozygous plants. The overexpressing lines were confirmed by Q-PCR using specific primers (APC10_Fwd tcatatccgccagatcaaagttt (SEQ ID NO:36) and APC10_Rev aaggttggtgcggaatagga (SEQ ID NO:37)) to confirm the mRNA levels of transgenic plants.

RNA Extraction and cDNA Preparation

Total RNA was extracted from the frozen materials using TRIzol Reagent (Invitrogen). To eliminate the residual genomic DNA present in the preparation, the RNA was treated by RNAse-free DNAse I according to the manufacturer's instructions (Amersham Biosciences) and purification with the RNEASY® Mini kit from Qiagen was performed. Total RNA was then quantified with a spectrophotometer and loaded onto an agarose gel to check its integrity. cDNA was made with “SuperScript III first strand synthesis system” (Invitrogen) with oligo (dT) primer solution on 2 μg RNA template according to the manufacturer's instructions.

### Proteolysis and Peptide Isolation

After destaining, gel slabs were washed for 1 hour in H₂O, polypeptide disulfide bridges were reduced for 40 minutes in 25 mL of 6.66 mM DTT in 50 mM NH₄HCO₃ and sequentially the thiol groups were alkylated for 30 minutes in 25 mL 55 mM IAM in 50 mM NH₄HCO₃. After washing the gel slabs three times with water, complete lanes from the protein gels were cut into slices, collected in microtiter plates and treated essentially as described before with minor modifications (Van Leene et al., 2007). Per microtiter plate well, dehydrated gel particles were rehydrated in 20 μL digest buffer containing 250 ng trypsin (M S Gold; Promega, Madison, Wis.), 50 mM NH₄HCO₃ and 10% CH₃CN (v/v) for 30 minutes at 4° C. After adding 10 μL of a buffer containing 50 mM NH₄HCO₃ and 10% CH₃CN (v/v), proteins were digested at 37° C. for 3 hours. The resulting peptides were concentrated and desalted with microcolumn solid phase tips (PerfectPure™ C18 tip, 200 nL bed volume; Eppendorf, Hamburg, Germany) and eluted directly onto a MALDI target plate (Opti-TOFTM384 Well Insert; Applied Biosystems, Foster City, Calif.) using 1.2 μL of 50% CH₃CN: 0.1% CF₃COOH solution saturated with α-cyano-4-hydroxycinnamic acid and spiked with 20 (mole/μL Glu1 Fibrinopeptide B (Sigma Aldrich), 20 fmole/μL des-Pro2-Bradykinin (Sigma Aldrich), and 20 fmole/μL Adrenocorticotropic Hormone Fragment 18-39 human (Sigma Aldrich).

### Acquisition of Mass Spectra

A MALDI tandem MS instrument (4700 and 4800 Proteomics Analyzer; Applied Biosystems) was used to acquire peptide mass fingerprints and subsequent 1 kV CID fragmentation spectra of selected peptides. Peptide mass spectra and peptide sequence spectra were obtained using the settings essentially as previously described (Van Leene et al., 2007). Each MALDI plate was calibrated according to the manufacturers' specifications. All peptide mass fingerprinting (PMF) spectra were internally calibrated with three internal standards at m/z 963.516 (des-Pro2-Bradykinin), m/z 1570.677 (Glu1-Fibrinopeptide B), and m/z 2465,198 (Adrenocorticotropic Hormone Fragment 18-39) resulting in an average mass accuracy of 5 ppm±10 ppm for each analyzed peptide spot on the analyzed MALDI targets. Using the individual PMF spectra, up to sixteen peptides, exceeding a signal-to-noise ratio of 20 that passed through a mass exclusion filter, were submitted to fragmentation analysis.

### MS-Based Protein Homology Identification

PMF spectra and the peptide sequence spectra of each sample were processed using the accompanied software suite (GPS Explorer 3.6, Applied Biosystems) with parameter settings essentially as previously described (Van Leene et al., 2007). Data search files were generated and submitted for protein homology identification against the TAIR 8.0 by using a local database search engine (Mascot 2.1, Matrix Science). Protein homology identifications of the top hit (first rank) with a relative score exceeding 95% probability were retained. Additional positive identifications (second rank and more) were retained when the score exceeded the 98% probability threshold.

### Flow Cytometry

Flow-cytometry analysis. The leaves' tissue were chopped with a razorblade in 200-400 μl of buffer (45 mM MgCl2, 30 mM sodium citrate, 20 mM 3-[N-morpholino]-propane-sulfonic acid, pH 7, and 1% Triton X-100), filtered over a 30 μm mesh, and 1 μl of 1 μg/mL of 4,6-diamidino-2-phenylindole (DAPI) was added. The nuclear DNA content distribution was analyzed with a Cyflow ML flowcytometer (Partec).

### Leaf Measurement and Cell Number Analysis

The leaf measurement and subsequent cell number analysis of SAMBA knockout and wild-type plants was performed on the abaxial epidermis of leaf 1 and 2 blades harvested on days 12 and 15, as described earlier (De Veylder et al., 2001). Plants were sown in quarter sections of round 12-cm Petri dishes filled with 100 mL of 0.5× Murashige and Skoog medium (Duchefa, Haarlem, The Netherlands) and 0.9% plant tissue culture agar. All healthy plants were placed in ethanol overnight to remove chlorophyll, and subsequently cleared and stored in lactic acid for microscopy. The complete kinematics analysis was performed as described earlier (De Veylder et al., 2001) on the abaxial epidermis of leaf 1 and 2 blades harvested daily from days 4 to 25 with APC10^{OE} and control plants.

### Phenotypic Analysis

For the biomass measurement, the vegetative part of a 20-day-old plant was harvested and the fresh weight was measured by weighing about 20 plants of each line and for dry weight, the same plants were placed on petri plates and allowed to dry for one week and weighed again. For the leaf area measurement, leaf series were made from plants grown in vitro for 22 days. Leaves were dissected from the rosettes on the left side, starting from two cotyledons followed from left to right by the 1^{st}, 2^{nd}, 3^{rd} and the subsequent leaves.

For the root analysis, the plants were grown on vertical position on plates with MS medium 1.2% agar during 15 days. After 15 days, the plates were scanned and the pictures were analyzed using image J 1.37 program. For fresh weight measurement, the total root of 25 plants was cut from the shoot and weighed individually and for dry weight, the same plants were placed on petri plates and allowed to dry for one week and weighed again

The seed size measurement was performed by placing the seeds on transparent plastic paper and each line was scanned separately. The images of total seed area were analyzed using image J 1.37 program.

### Kinematic Analysis

Kinematic analysis was performed as described earlier (De Veylder et al., 2001) on the abaxial epidermis of leaf 1 and 2 blades harvested daily from days 4 to 25.

### Mannitol Experiment

Seedlings of SAMBA knockout and wild-type. Ecotype Columbia-0 (Col-0) were grown in vitro in half-strength Murashige and Skoog medium (Murashige and Skoog, 1962), supplemented with 1% sucrose under a 16-hour day (110 μmol m-2 s-1) and 8-hour night regime. Before autoclaving, 25 mM Mannitol (Sigma) was added to the agar medium. The treated plants were grown on 25 mM Mannitol plates, while the control plants were grown on the same medium without Mannitol. The plants were grown during 20 days and the pictures were taken and the images were analyzed using Image J 1.37 program.

### Example 1

### Effect of APC10 on Plant Growth

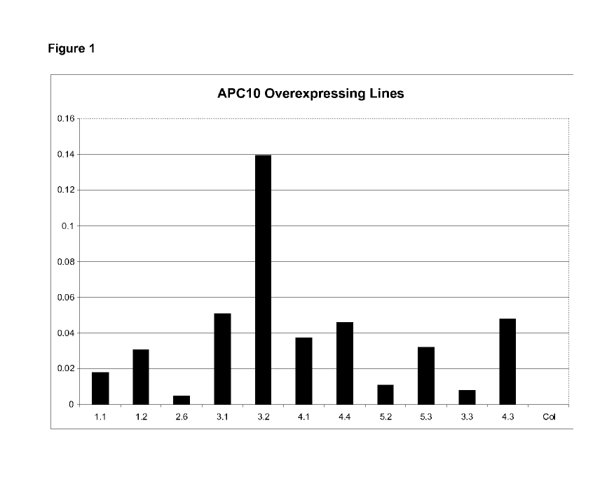

To assess the function of APC10 during development, Arabidopsis plants expressing higher levels of APC10 mRNA under the control of the cauliflower mosaic virus (CaMV) 35S constitutive promoter were generated. We selected 11 independent homozygous, single locus plants in which the increased expression levels of APC10 was confirmed by QPCR (FIG. 1).

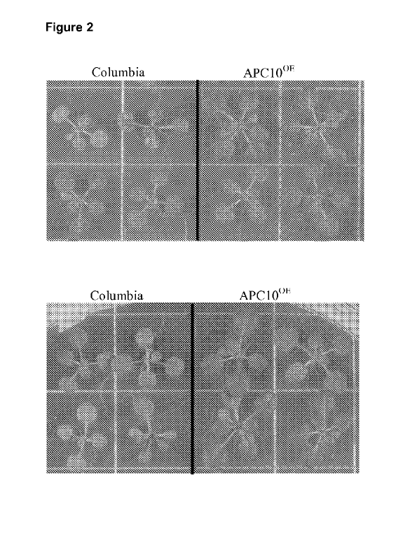

Comparative phenotype analyses between APC10 overexpressing lines (APC 10^{OE}) and control lines showed that plants with higher levels of APC 10 caused an increase in the rosette and leaf growth during development (FIG. 2).

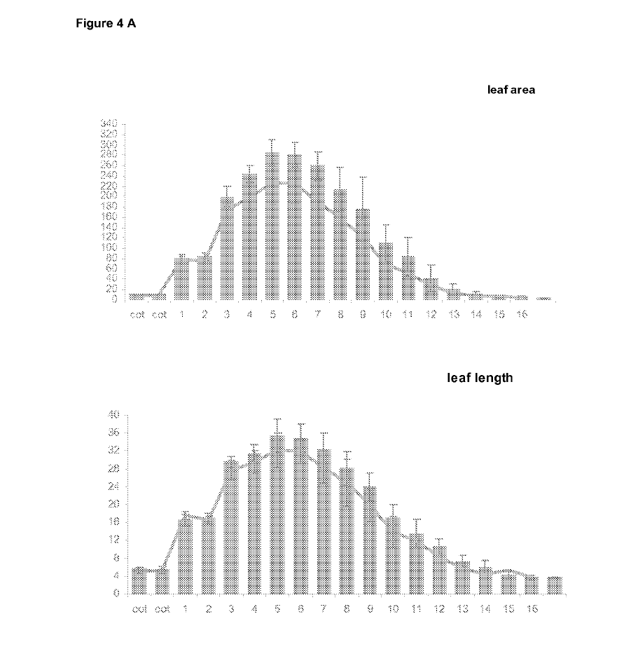

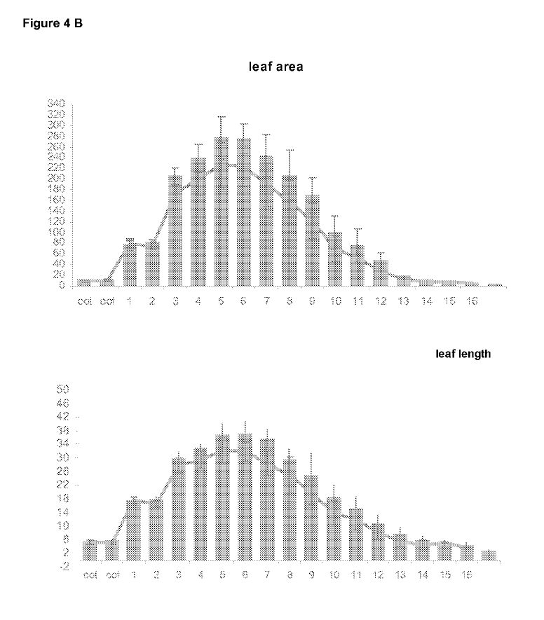

To know which of the leaves were affected, we determined the area of all leaves from two independent lines from APC10^{OE} and wild-type control. In three-week-old grown in the soil, the area of all leaves was significantly increased in the transgenic plants when compared to wild-type controls (FIG. 4).

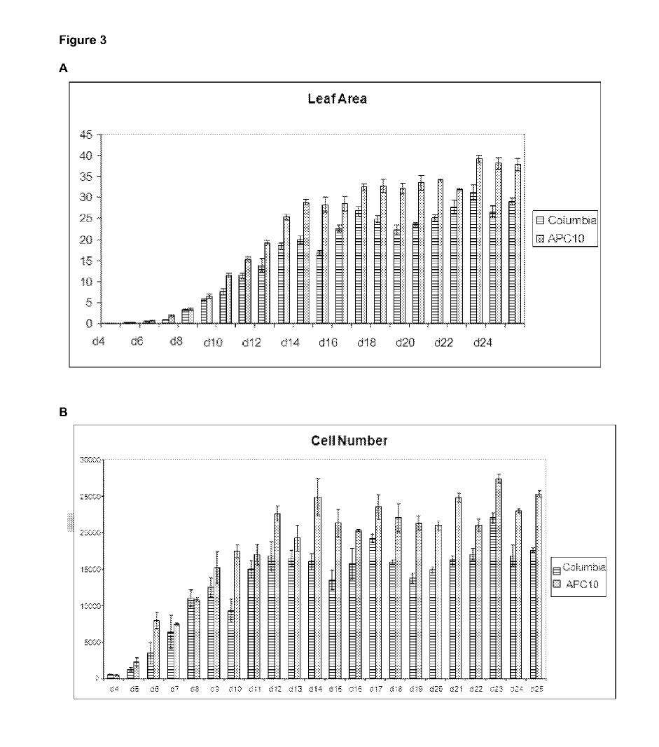

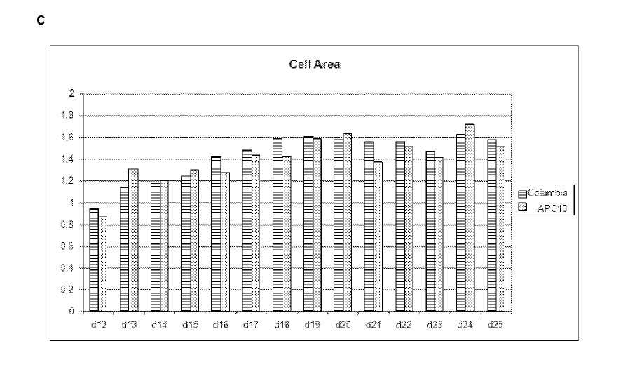

To investigate the cellular basis of the observed phenotype, we performed kinematics analysis of developing leaves. FIG. 3 show a significantly increased leaf area and cell number in APC10^{OE} plants from the beginning of development (day 4 and day 5) when compared to wild-type plants.

The main conclusion is that cell division rates were higher in APC10^{OE} plants during early leaf development when compared with wild-type controls. Though leaf cell organization and cell sizes were similar to those of control plants, cell numbers were significantly increased in mature leaves of APC10^{OE} plants.

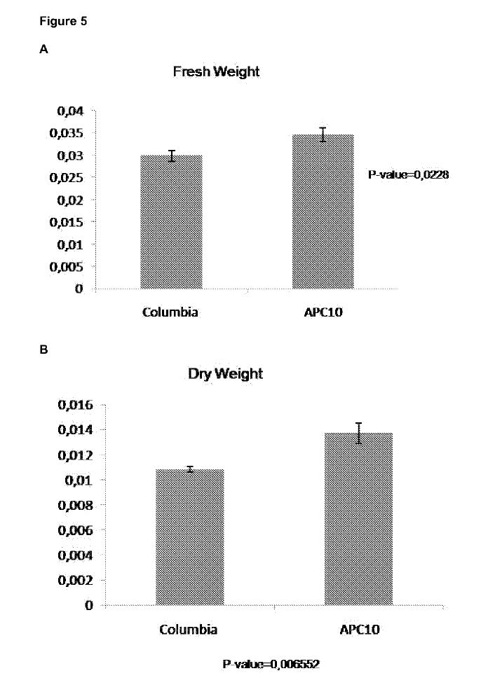

To verify if we have significant difference on biomass of transgenic plants compared to wild-type, the fresh and dry weights of shoots were measured in APC10^{OE} and wild-type plants. We observed an increase of biomass in the transgenic plants when compared to wild-type controls (FIG. 5, Panels A and B), the fresh and dry weight of those plants were about 15% higher than wild-type plants.

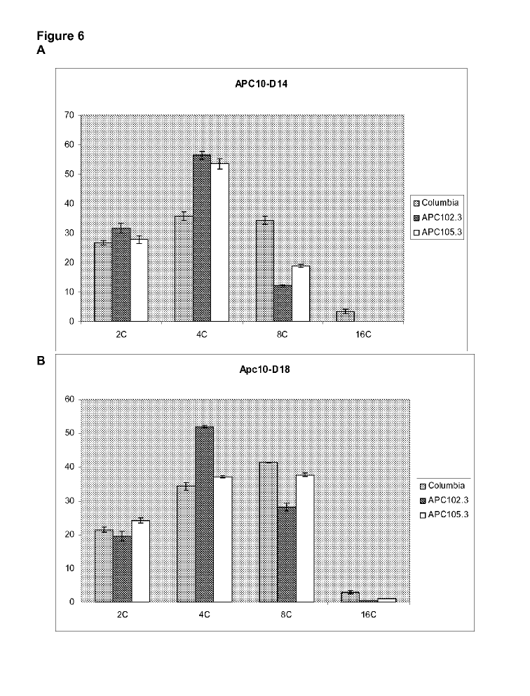

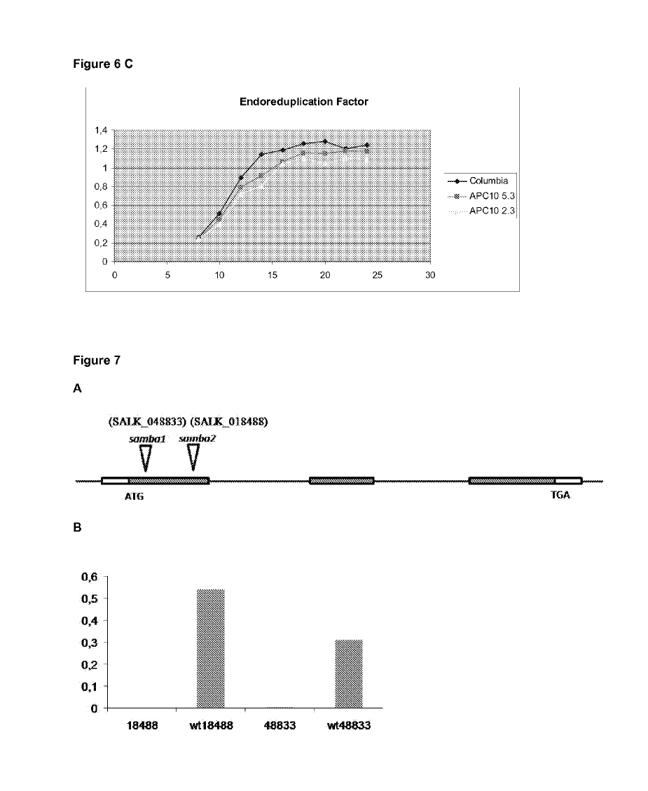

We analyzed the DNA content in different developmental stages: proliferation (d8; d10 and d12), expansion (d14; d16 and 18), and mature tissues (d20; d22; d24) of leaf cells of the APC10^{OE} plants. We observed a higher proportion of cells with 2C and 4C DNA contents and, conversely, a lower proportion of cells with 8C and 16C DNA contents compared to wild-type plants, showing that in APC10^{OE} plants, the endoreduplication is reduced (FIG. 6).

### Example 2

### TAP Isolation and MS Identification of APC10 Interacting Proteins

In order to identify the interaction partners of APC10 in vivo, we performed tandem affinity (TAP) purifications on transgenic Arabidopsis cell suspension cultures that expressed under control of the 35ScaMV promoter the APC10 as a protein fused at its N-terminus with the traditional TAP tag developed for yeast (Rigaut et al., 1999) and with the GS tag (Bürckstümmer et al., 2006). Four independent TAP purifications were performed on the cultures with the traditional tag according to Van Leene et al. (2007), and two purifications on the cultures with the GS tag according to Van Leene et al. (2008). Protein extracts were harvested two days after sub-culturing into fresh medium. The affinity-purified proteins were separated on a 4-12% NuPAGE gel and stained with Coomassie Brilliant Blue. Protein bands were cut, in-gel digested with trypsin and subjected to MALDI-TOF/TOF mass spectrometry for protein identification. After subtracting background proteins, identified by control purifications (Van Leene et al., 2007 and 2008), we identified 18 APC10 interacting proteins (Table 2). These can be divided into two groups: 14 proteins were confirmed experimentally and four proteins were identified only in one out of six TAP experiments and which may represent rather weak or transient interactions.

### Example 3

### Stimulation of Plant Growth by a Novel APC Interactor (SAMBA) Protein Knockout

Among the interacting proteins, a novel 100-amino-acid protein (AT1G32310) was identified (Table 2). We selected this protein to analyze in more detail because it showed very specific binding with APC 10 subunit. The expressed protein is an unknown protein similar to unknown protein from Oryza sativa (GB:AAL67597.1).

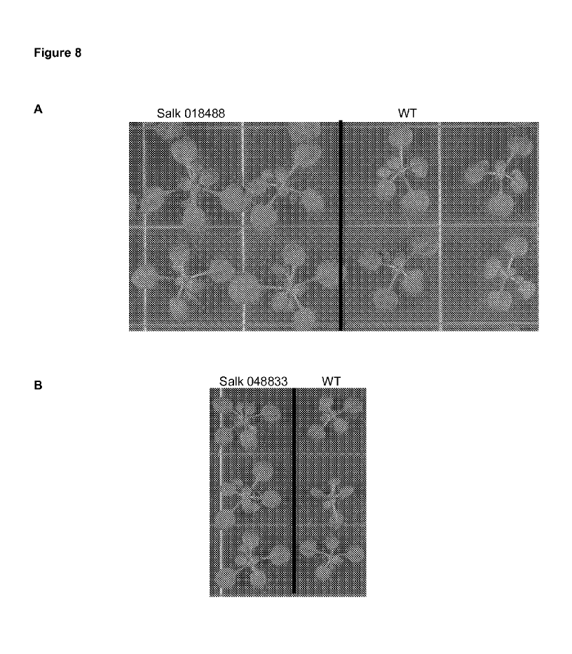

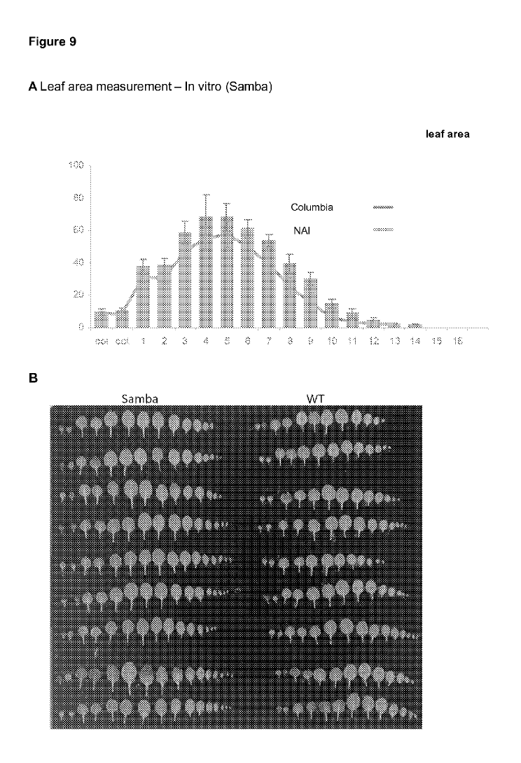

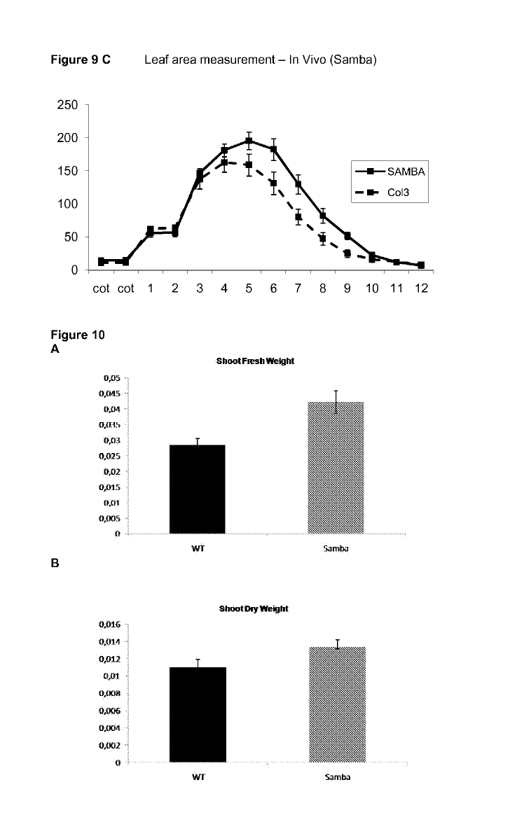

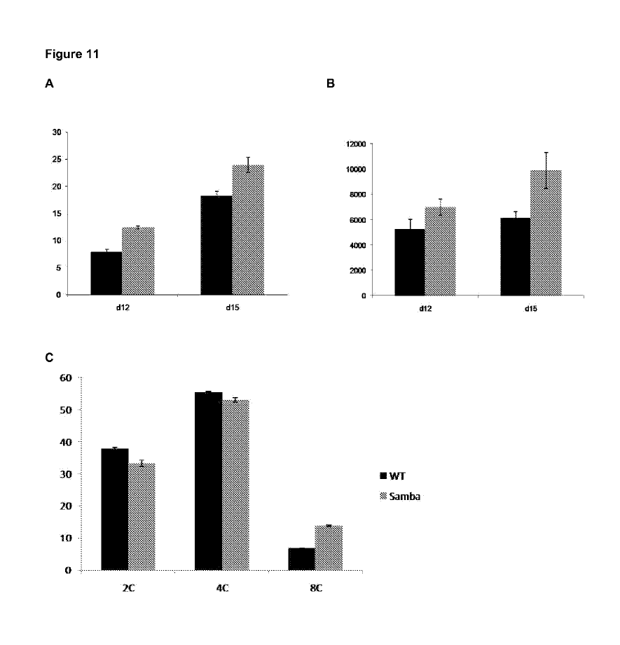

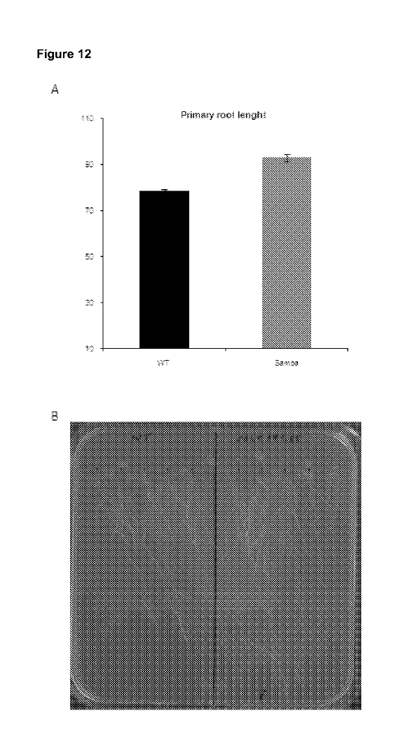

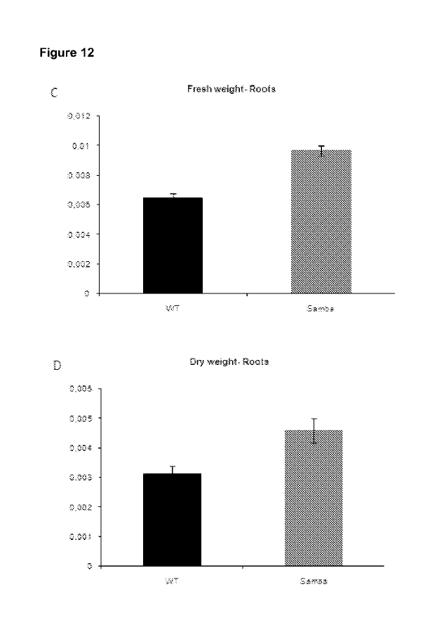

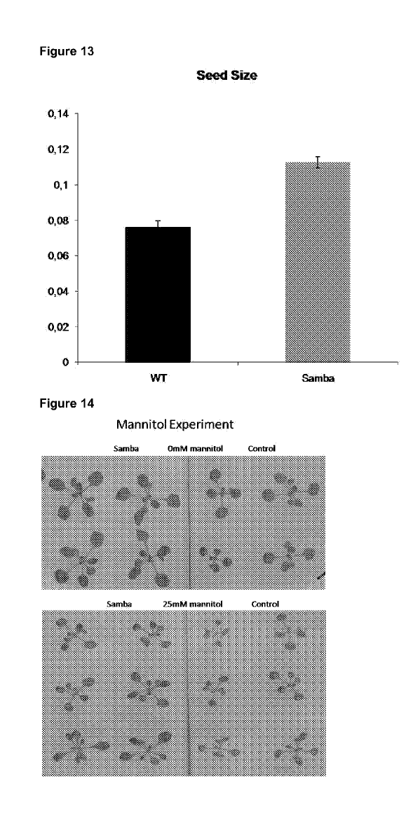

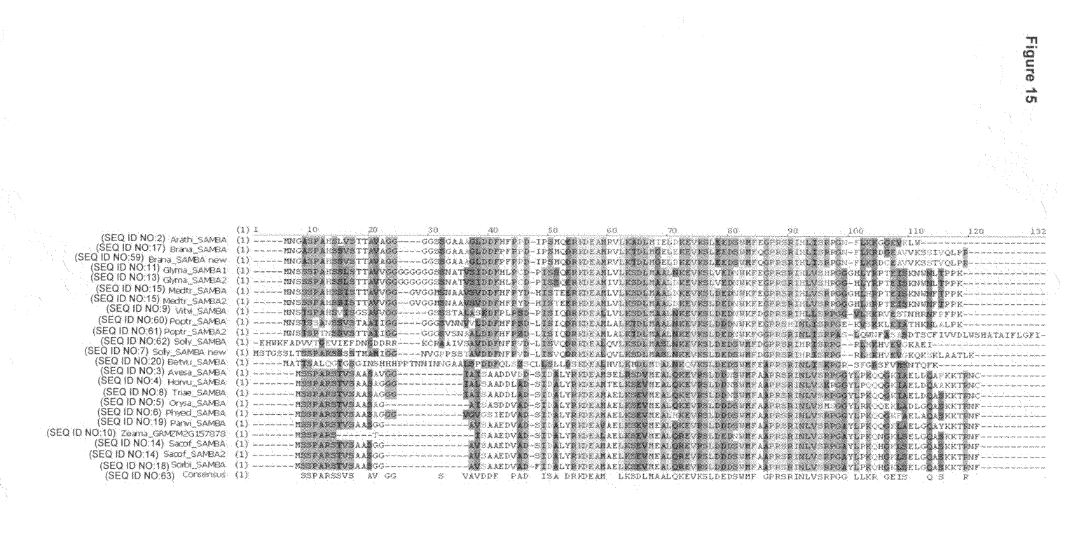

To better understand the function of this gene, knockout plants from SALK collection were selected and analyzed. The representative scheme of T-DNA insertions on the first exon of SAMBA gene is shown in FIG. 7, Panel A. SAMBA transcripts were not detected in the SAMBA mutant plants by Q-PCR analysis (FIG. 7, Panel B), confirming the loss of function of the gene. The mutant plants (homozygous SALK lines) of the SAMBA knockouts showed an increase in the rosette and in the leaf growth when compared to wild-type controls (FIG. 8) similar to the APC10^{OE} plants phenotype. The measurement of total leaf area of SAMBA mutants grown in vitro also showed a significant increase in the leaf area compared to wild-type plants (FIG. 9). The measurement of fresh and dry weight shoots (FIG. 10), leaf area (FIG. 11), root length and weight (FIG. 12) and seed size (FIG. 13) all showed a significant increase for the SAMBA knockout plants, proving that SAMBA is a new gene controlling the growth of plants.

The phenotype of the SAMBA knockout was analyzed in detail by measuring the total leaf area of 22-day-old plants grown in vitro. The result showed a significantly increased leaf area compared to wild-type plants (FIG. 9, Panel A). The same analysis was made with 22-day-old plants grown on soil and we could observe the same phenotype of plants grown in vitro, a significantly increased leaf area in SAMBA knockout compared to wild-type plants (FIG. 9, Panel C). To verify if there was a significant difference on biomass of SAMBA knockout compared to wild-type plants, we measured the fresh and dry weight of the vegetative part of 20-day-old plants. The measurement of fresh (FIG. 10, Panel A) and dry weight (FIG. 10, Panel B) showed a significant increase in the biomass of SAMBA knockout plants, corroborating with the hypothesis of a new candidate gene controlling the growth of plants.

To investigate the cellular basis of the observed phenotype, we measured and analyzed the area and cell number of the first pair of leaves of day 12 and 15. FIG. 11, Panels A and B, show a significantly increased leaf area and cell number in SAMBA knockout compared to wild-type plants, indicating that cell division is higher in SAMBA knockout plants.

Flow cytometry analysis was performed to analyze the impact of reduced expression of the SAMBA gene on the plant DNA content. The SAMBA knockout plants show slight increased levels of 8C DNA content when compared to wild-type plants (FIG. 11, Panel C).

The impact of the SAMBA knockout on root and seed yield was also evaluated. The primary root length was measured 15 days after germination. The data show a significant increase on the length of SAMBA knockout roots compared to wild-type plants (FIG. 12, Panel A). The representative picture of longer roots of SAMBA knockout is shown in the FIG. 12, Panel B. The fresh and dry weight (FIG. 12, Panels C and D) of roots were measured and we can confirm a significant increase of root biomass in SAMBA knockout plants.

The analysis of seed also shows an increased seed size. The total seed area of plants, wild-type and SAMBA mutant, was measured. As we can observe, the seed of SAMBA mutants are significantly bigger than wild-type plants (FIG. 13).

### Example 4

### Effect of the SAMBA Knockout Under Stress Conditions

Wild-type and SAMBA knockout plants were grown on agar plates supplemented with 25 mM Mannitol to evaluate the capacity of SAMBA mutant plants to grow under stress conditions. As shown in FIG. 14, the SAMBA mutant plants keep their increased biomass phenotype under stress conditions.

TABLE 1
Non-limiting examples of target
sequences for RNAi

| Arabidopsis thaliana | |
|---|---|
| TAAACAAAGCGTATATGACCA | (SEQ ID NO: 38) |
| TCATTTTCGAGTAATAGGCTC | (SEQ ID NO: 39) |
| Hordeum vulgare | |
| TAAGTTATGACTTATGAGCAT | (SEQ ID NO: 40) |
| TTTAGATGAATGCAACTCCAT | (SEQ ID NO: 41) |
| Oryzae sativa | |
| TAGAATTCTACCAGGCGTCTT | (SEQ ID NO: 42) |
| TTGAGTAATCCTTACATGCGA | (SEQ ID NO: 43) |
| Brassica napus | |
| TATAAAGTTCGTGATGGACAT | (SEQ ID NO: 44) |
| TACTAGATATCACCAAACCTA | (SEQ ID NO: 45) |
| Saccharum officinarum | |
| TTCTACACCCTAGAAGTTCTT | (SEQ ID NO: 46) |
| TACTAGGCTTCTTACAAGCAC | (SEQ ID NO: 47) |
| Glycine max | |
| TATCAAGCTTTAAGTGTGCTC | (SEQ ID NO: 48) |
| TTAACATGACACGAACTTCGC | (SEQ ID NO: 49) |
| Vinis vinifera | |
| TCTTGTGGAGAACTCCCCCAG | (SEQ ID NO: 50) |
| TCTTGTGGAGAACTCCAGCAG | (SEQ ID NO: 51) |
| Solanaceum lycopersicum | |
| TATCTATACTCGTTATCGCAC | (SEQ ID NO: 52) |
| TATCTATACTCGTAATCGCTC | (SEQ ID NO: 53) |
| TATCTCATATGGAATTCGCGC | (SEQ ID NO: 54) |
| Tricitum aestivum | |
| TTAACAGGTGAGTCGAATCAG | (SEQ ID NO: 55) |
| TTAACAGGTGAGTCGAATCAT | (SEQ ID NO: 56) |
| Zea mays | |
| TCAACTCTGAGAGTTTCGCAT | (SEQ ID NO: 57) |
| TTACCATGACATTAACGTCGC | (SEQ ID NO: 58) |

TABLE 2
List of APC10-copurified proteins identified by MS.

| | | | | | Sequence | Protein | Best Ions |
|---|---|---|---|---|---|---|---|
| Atnumber_{—} | | Found/ | | Peptide | coverage | Score/ | Score/ |
| Prey | Prey | 6 exp | Mass | count | % | Threshold | Threshold |
| AT2G39090 | APC7 | 6 | 57877 | 29 | 68% | 1420/58 | 135/25 |
| AT2G20000 | CDC27b | 6 | 83756 | 23 | 41% | 1200/58 | 123/25 |
| AT5G05560 | APC1 | 6 | 188495 | 32 | 29% | 851/58 | 109/26 |
| AT3G48150 | APC8 | 6 | 67776 | 21 | 46% | 574/58 | 91/24 |
| AT1G06590 | APC5 | 6 | 101945 | 21 | 38% | 567/58 | 71/25 |
| AT1G78770 | CDC16 | 6 | 62862 | 11 | 24% | 265/58 | 129/20 |
| AT4G21530 | APC4 | 6 | 88330 | 16 | 27% | 215/58 | 54/22 |
| AT2G04660 | APC2 | 3 | 98470 | 13 | 18% | 177/58 | 77/24 |
| AT1G32310 | SAMBA | 4 | 10849 | 2 | 25% | 134/58 | 82/21 |
| AT2G42260 | UVI4 | 3 | 28713 | 6 | 29% | 114/58 | 54/27 |
| AT4G19210 | RNase L inhibitor | 2 | 69202 | 8 | 16% | 102/58 | 32/25 |
| | protein, putative | | | | | | |
| AT3G57860 | UVI4-like | 2 | 27092 | 5 | 28% | 95/58 | 72/21 |
| AT3G16320 | CDC27a | 4 | 82032 | 4 | 8% | 63/58 | 53/25 |
| AT4G25550 | expressed protein | 2 | 23043 | 3 | 17% | 50/58 | 37/24 |
| AT5G13840 | CCS52B | 1 | 52915 | 9 | 24% | 87/58 | 28/24 |
| AT3G48750 | CDKA; 1 | 1 | 34123 | 9 | 31% | 60/58 | |
| AT3G56150 | eukaryotic translation | 1 | 103283 | 9 | 10% | 57/58 | 29/27 |
| | initiation factor 3 | | | | | | |
| | subunit 8 | | | | | | |
| AT2G06210 | phosphoprotein-related | 1 | 121256 | 9 | 9% | 53/58 | 34/26 |
| | (ELF8) | | | | | | |

The third column mentions in how many of the six independent experiments an interactor was identified.

### REFERENCES

Altschul, S. F., T. L. Madden, A. A. Schäffer, J. Zhang, Z. Zhang, W. Miller, and D. L. Lipman (1997). Gapped BLAST and PSI-BLAST: a new generation of protein database search programs. Nucleic Acids Res. 25:3389-3402.
Altschul, S. F., J. C. Wootton, E. M. Gertz, R. Agarwala, A. Morgulis, A. A. Schäffer, and Y. K. Yu (2005). Protein database searches using compositionally adjusted substitution matrices. FEBS J. 272:5101-5109.
Au, S. W., X. Leng, J. W. Harper, and D. Barford (2002). Implications for the ubiquitination reaction of the anaphase promoting complex from the crystal structure of the Doc1/Apc10 subunit. J. Mol. Biol. 316:955-968.
Bürckstümmer, T., K. L. Bennett, A. Preradovic, G. Schütze, O. Hantschel, G. Superti-Firga, and A. Bauch (2006). An efficient tandem affinity purification procedure for interaction proteomics in mammalian cells. Nat. Methods 3:1013-1019.
Carrol, C. W., E. M. Newman, and D. O. Moragn (2005). The APC subunit Doc1 Promotes Recognition of the substrate Destruction Box. Current Biol. 15:11-18.
Clough, S. J., and A. F. Bent (1998). Floral dip: A simplified method for Agrobacterium-mediated transformation of Arabidopsis thaliana. Plant J. 16:735-743.
De Veylder, L., T. Beeckman, G. T. S. Beemster, L. Krols, F. Terras, I. Landrieu, E. Van der Schueren, S. Maes, M. Naudts, and D. Inzé (2001). Functional analysis of cyclin-dependent kinase inhibitors of Arabidopsis. Plant Cell 13:1653-1668.
Eloy, N. B., F. Coppens, G. T. S. Beemster, A. S. Hemerly, and P. C. G. Ferreira (2006). The Arabidopsis Anaphase Promoting Complex (APC): Regulation Through Subunit Availability in Plant Tissues Cell Cycle 5:17, 1957-1965. 2006
Hershko, A., and C. Ciechanover (1998). The ubiquitin system. Annu. Rev. Biochem. 67:425-79.
Morgan, D. O. (1999). Regulation of the APC and exit from Mitosis. Nat. Cell. Biol. 1:E47-E53.
Ossowski, S., J. Fitz, R. Schwab, M. Riester, and D. Weigel (© Copyright 2005-2009) Max Planck Institute for Developmental Biology, Tübingen. http://www.weigelworld.org
Passmore, L. A., E. A. McCormack, S. W. Au, A. Paul, K. R. Willison, J. W. Harper, and D. Barford (2003). Docl mediates the activity of the anaphase-promoting complex by contributing to substrate recognition. EMBO J. 22:786-796.
Peters, J. M. (2002). The anaphase-promoting complex: Proteolysis in mitosis and beyond. Molecular Cell 9:931-43.
Peters, J. M., R. W. King, C. Hoog, and M. W. Kirschner (1996). Identification of BIME as a subunit of the anaphase-promoting complex. Science 274:1199-201.
Rigaut, G., A. Shevchenko, B. Rutz, M. Wilm, M. Mann, and B. Seraphin (1999). A generic protein purification method for protein complex characterization and proteome exploration. Nat. Biotechnol. 17:1030-1032.
Van Leene, J., D. Eeckhout, H. Stals, G. Persiau, E. Van De Slijke, G. Van Isterdael, K. Laukens, N. Remmerie, A. Abdelkrim, A. Pharazyn, H. Van Onckelen, D. Inzé, E. Witters, and G. De Jaeger (2007). Tandem affinity purification of cell cycle protein complexes from Arabidopsis cell suspension cultures. Mol. Cell Proteomics 6:1226-1238.
Van Leene, J., H. Stals, D. Eeckhout, G. Persiau, E. Van De Slijke, G. Van Isterdael, A. De Clercq, E. Bonnet, K. Laukens, N. Remmerie, K. Henderickx, T. De Vijlder, A. Abdelkrim, A. Pharazyn, H. Van Onckelen, D. Inzé, E. Witters, and G. De Jaeger (2007). A tandem affinity purification-based technology platform to study the cell cycle interactome in Arabidopsis thaliana. Mol. Cell Proteomics 6:1226-1238.
Van Leene, J., E. Witters, D. Inzé, and G. De Jaeger (2008). Boosting tandem affinity purification of plant protein complexes. Trends Plant Sci. 13:517-520.
Yoon, H. J., A. Feoktistova, B. A. Wolfe, J. L. Jennings, A. J. Link, and K. L. Gould (2002). Proteomics analysis identifies new components of the fission and budding yeast anaphase-promoting complexes. Curr. Biol. 12:2048-54.

1. A method of increasing growth and/or yield of a plant, the method comprising:
utilizing, with the plant, APC10, or a variant thereof, and/or an APC10 interacting protein to increase plant growth and/or yield.

2. The method according to claim 1, wherein said interacting protein selected from the group consisting of AT2G39090, AT2G20000, AT5G05560, AT3G48150, AT1G06590, AT1 G78770, AT4G21530, AT2G04660, AT1G32310, AT2G42260, AT4GA19210, AT3G57860, AT3G16320, AT4G25550, AT5G13840, AT3G48750, AT3G56150 and AT2G06210.

3. The method according to claim 1, wherein said interacting protein comprises SEQ ID NO:2 or a variant thereof.

4. The method according to claim 3, wherein a variant is utilized, and said variant comprises SEQ ID NO:22 and/or SEQ ID NO:23.

5. The method according to claim 3, wherein a variant is utilized, and said variant comprises SEQ ID NO:26.

6. The method according to claim 1, wherein APC10 or a variant thereof is overexpressed.

7. The method according to claim 3, wherein the expression of SEQ ID NO:2 or the variant thereof is repressed.

8. A method of increasing growth and/or yield of a plant, the method comprising:
utilizing, with the plant, an RNAi against a nucleic acid molecule encoding SEQ ID NO:2, or a variant thereof, to increase plant growth and/or yield.

9. The method according to claim 1, wherein said plant is a crop plant.

10. The method according to claim 9, wherein said crop plant is a cereal plant.

11. The method according to claim 10, wherein said cereal plant is selected from the group consisting of rice, maize, wheat, barley, millet, rye, sorghum and oats.

12. The method according to claim 1, wherein the increase of plant growth is any one or more of an increase in leaf biomass, an increase in root biomass, and an increase in seed biomass.

13. A transgenic plant, comprising an RNAi against a nucleic acid encoding SEQ ID NO:2 or a variant thereof.

14. The transgenic plant according to claim 13, wherein said transgenic plant is a cereal plant.

15. The transgenic plant according to claim 14, wherein said cereal plant is selected from the group consisting of rice, maize, wheat, barley, millet, rye, sorghum and oats.

16. A method of increasing growth and/or yield of a plant, the method comprising:
over-expressing APC10 and/or repressing the expression of SEQ ID NO:2 in the plant,
so as to increase plant growth and/or yield.

17. The method according to claim 16, wherein the plant is a cereal plant selected from the group consisting of rice, maize, wheat, barley, millet, rye, sorghum, and oat.

18. The method according to claim 16, wherein increase of plant growth and/or yield comprises an increase in leaf biomass of the plant, an increase in root biomass of the plant, and an increase in seed biomass of the plant.


SEQUENCE LISTING (58 sequences)

SEQ ID NO: 1 - protein, 192 aa, Arabidopsis thaliana
MATESSESEEEGKISGGNYKLIIDDDLREMGKNAAWSVSSCKPGNGVTTLRDDNLETYWQSDGLQPHLINIQFQKKVKLQLVVLYVDFKLDESYTPSKISIRAGDGFHNLKEIKSVELVKPTGWVCLSLSGTDPRETFVNTFMLQIAILSNHLNGRDTHIRQIKVYGPRPNPIPHQPFQFTSMEFLTYSTLR

SEQ ID NO: 2 - protein, 100 aa, Arabidopsis thaliana
MNGASPAHSLVSTTAVAGGGGSSGAAAGLDDFHFPPDIPSMQERKDEAMRVLKADLMTELDKEVKSLEEDSWMFEGPRSRIHLISRRGNFLKKGGEVKLW

SEQ ID NO: 3 - protein, 102 aa, Avena sativa
MSSPARSTVSAASAVGGIAISAADDVDDSIDALYRKDEAMSELRSDVMEALQKEVRSLDDNSWMFAAPRSRINLVSRPGGYLPKQQGKIAELDQAPKKTRNC

SEQ ID NO: 4 - protein, 102 aa, Hordeum vulgare
MSSPARSTVSAASAGGGIALSAADDLADSIDALYRKDEAMTELKSEVMEALQKEVRSLDDNSWMFAAPRSRINLVSKRGGYLPQQQGKIAELDQAAKKTRNC

SEQ ID NO: 5 - protein, 99 aa, Oryza sativa
MSSPARSTVSAASAGAISASDDVADSIDALYRKDEAMAELKSEVMEALQKEVRSLDDDSWMFAAPRSRINLVSMSGGYLRKQQEKLADLGQASKKTRNF

SEQ ID NO: 6 - protein, 102 aa, Phyllostachys edulis
MSSPARSTVSAASAGGGVGVGSIEDVADSIDALYRKDEAMAELKSEVMEALHKEVRSLDDDSWMFAAPRSRINLVSRPGGYLPKQQGKFAELAQASKKTRNF

SEQ ID NO: 7 - protein, 113 aa, Solanum lycopersicon
MSTGSSLTSSPARSSSSTMAMIGGNVGPPSSTAVDDFNFPVDLISVQDRKDEALQVLKSDLMASLNKEVKSLDEDSWMFDGPRSRIHRISRPGRLHKHVEVGKQKSKLAATLK

SEQ ID NO: 8 - protein, 102 aa, Triticum aestivum
MSSPARSTVSAASAGGGIAISAADDLADSIDALYRKDEAMAELKSEVMEALQKEVRSLDDNSWMFAAPRSRINLVSRPGGYLPKQQGKIAELDQASKKTRNC

SEQ ID NO: 9 - protein, 106 aa, Vitis vinifera
MNSTSPAHSVISGSAVVGGGSSSTALASEDFPLPSDPISIQDRKDEALLVLKSDLMAALNKEVKSLDEDNWKFDGPRSRIHLLSRPGGVLHKRVESTNHRNFPFPK

SEQ ID NO: 10 - protein, 91 aa, Zea mays
MSSPARSTISAAEDVADSIDALYRKDEALAELKSEVMEALQREVRSLDEDNWMFAAPRSRINLVSRPGAYLPKQNGKLSELGQASKKTRNF

SEQ ID NO: 11 - protein, 112 aa, Glycine max
MNSSSPAHSSLSTTAVVGGGGGGGGSSNATVSIDDFHLPCDPISSQERKDEAMLVLKSDLMAALNKEVKSLVEDNWKFEGPRSRIHLVSHRGGGHLYRPTEISKNWNLTPPK

SEQ ID NO: 12 - protein, 111 aa, Glycine max
MNSSSPAHSSLSTTAVVGGGGGGGGSSNATVSIDDFHLPCDPISSQERKDEAMLVLKSDLMAALNKEVKSLVEDNWKFEGPRSRIHLVSHRGGHLYRPTEISKNWNLTPPK

SEQ ID NO: 13 - protein, 111 aa, Glycine max
MNSSSPAHSSLSTTAVVGGGGGGGGSSNATVSIDDFHLPCDPISSQERKDEAMIVLKSDLMAALDKEVKSLVEDNWKFEGPRSRIHLVSHRGGHLYRPTEISKNWNLTPPK

SEQ ID NO: 14 - protein, 99 aa, Saccharum officinarum
MSSPARSTVSAASGGAVSAAEDVADSIDALYRKDEAMAELKSEVMEALQREVRSLDDDSWMFAAPRSRINLVSRPGAYLPKQHGKLSELGQASKKTRNF

SEQ ID NO: 15 - protein, 110 aa, Medicago truncatula
MNSSSPAHSSISTTAVVGGGVGGMSNAAVSVDDFHFPYDHISTEERKDEAMLVLKSDLMAALDKEVKSLDEDNWKFEGPRSRIHLVSRRGGGHLHRPTEISKNWNFTPPK

SEQ ID NO: 16 - protein, 107 aa, Populus tremula
MNSTSPTNSSVSTTAIIGGGGGSVSNAALDEFNFPSDLISIQDRKDEAMLALKTDLMAALNKEVKSLDEDNWKFEGPRSRINHISRPGGFLSKKLEITKKKNLALPK

SEQ ID NO: 17 - protein, 108 aa, Brassica napus
MNGASPAHSSVSTTAVAGGGGSSGAAAGLDDFPFPPDIPSMQDRKDEAMRVLKTDLMGELEKEVKSLEEDSWMFQGPRSRIHLISRRGNFLKRDGEAVVKSSIVQLPR

SEQ ID NO: 18 - protein, 99 aa, Sorghum bicolor
MSSPARSTVSAASGGAVSAAEDVADFIDALYRKDEAMAELKSEVMEALQREVRSLDDDSWMFAAPRSRINLVSRPGAYLPKQHGKLSELGQASKKTRNF

SEQ ID NO: 19 - protein, 99 aa, Panicum virgatum
MSSPARSTVSAASGGAVSAAEDVADSIDALYRKDEAVAELKSEVMEALQKEVRSLDDDSWMFAAPRSRINLVSRPGAYLPKQQGKLAELGQAYKKTRNF

SEQ ID NO: 20 - protein, 108 aa, Beta vulgaris
MATTSALQGTGSGINSHHHPPTNNINNGAALSPDDFQLSSSQLLSLLDSKDEALHVLKMDLMDALNKQVKSLDEDSWMFEAPRSRINLISKPGRSFGRSFVMSNTQFK

SEQ ID NO: 21 - protein, 121 aa, Ricinus communis
MNSTSPAHSSVSTTAIVGGGSVTNPSFDEFQFPSDLISIHDRKEEAFLVLKADLMVTLNKEVKSLDEDNWKFEGPRSCIHLISRPGGFPNKKMEITKSRNLVQPKNNSNSSDRSCLSSVDY

SEQ ID NO: 22 - protein, 4 aa, Arabidopsis thaliana
  MISC_FEATURE (2)..(2): X can be D or E
KXEA

SEQ ID NO: 23 - protein, 5 aa, Arabidopsis thaliana
  MISC_FEATURE (4)..(4): X can be R, H or C
PRSXI

SEQ ID NO: 24 - protein, 6 aa, Arabidopsis thaliana
  MISC_FEATURE (1)..(1): X can be R or S
XKXEAX

SEQ ID NO: 25 - protein, 8 aa, Arabidopsis thaliana
  MISC_FEATURE (2)..(2): X can be E, Q, D, G or A
FXXPRSXI

SEQ ID NO: 26 - protein, 37 aa, Arabidopsis thaliana
  MISC_FEATURE (2)..(2): X can be D or E
KXEAXXXLXXXXMXXLXXXVXXLXXXXWXFXXPRSXI

SEQ ID NO: 27 - DNA, 56 bp, Artificial Sequence
  Primer
ggggacaagtttgtacaaaaaagcaggcttcacaatggcgacagagtcatcggaat

SEQ ID NO: 28 - DNA, 53 bp, Artificial Sequence
  Primer
ggggaccactttgtacaagaaagctgggtatgttcttcaaacttctcctgctc

SEQ ID NO: 29 - DNA, 21 bp, Artificial Sequence
  Primer
atgacgaaacaccgaaaacac

SEQ ID NO: 30 - DNA, 21 bp, Artificial Sequence
  Primer
agttttatggtcggtcacacg

SEQ ID NO: 31 - DNA, 21 bp, Artificial Sequence
  Primer
ccattgggatcattactgctg

SEQ ID NO: 32 - DNA, 21 bp, Artificial Sequence
  Primer
aaaggaaacgtgacgattgtg

SEQ ID NO: 33 - DNA, 19 bp, Artificial Sequence
  Primer
attttgccgatttcggaac

SEQ ID NO: 34 - DNA, 20 bp, Artificial Sequence
  Primer
gctggtctagacgatttcca

SEQ ID NO: 35 - DNA, 20 bp, Artificial Sequence
  Primer
gcttcacttcacctcctttc

SEQ ID NO: 36 - DNA, 23 bp, Artificial Sequence
  Primer
tcatatccgccagatcaaagttt

SEQ ID NO: 37 - DNA, 20 bp, Artificial Sequence
  Primer
aaggttggtgcggaatagga

SEQ ID NO: 38 - DNA, 21 bp, Arabidopsis thaliana
taaacaaagcgtatatgacca

SEQ ID NO: 39 - DNA, 21 bp, Arabidopsis thaliana
tcattttcgagtaataggctc

SEQ ID NO: 40 - DNA, 21 bp, Hordeum vulgare
taagttatgacttatgagcat

SEQ ID NO: 41 - DNA, 21 bp, Hordeum vulgare
tttagatgaatgcaactccat

SEQ ID NO: 42 - DNA, 21 bp, Oryza sativa
tagaattctaccaggcgtctt

SEQ ID NO: 43 - DNA, 21 bp, Oryza sativa
ttgagtaatccttacatgcga

SEQ ID NO: 44 - DNA, 21 bp, Brassica napus
tataaagttcgtgatggacat

SEQ ID NO: 45 - DNA, 21 bp, Brassica napus
tactagatatcaccaaaccta

SEQ ID NO: 46 - DNA, 21 bp, Saccharum officinarum
ttctacaccctagaagttctt

SEQ ID NO: 47 - DNA, 21 bp, Saccharum officinarum
tactaggcttcttacaagcac

SEQ ID NO: 48 - DNA, 21 bp, Glycine max
tatcaagctttaagtgtgctc

SEQ ID NO: 49 - DNA, 21 bp, Glycine max
ttaacatgacacgaacttcgc

SEQ ID NO: 50 - DNA, 21 bp, Vitis vinifera
tcttgtggagaactcccccag

SEQ ID NO: 51 - DNA, 21 bp, Vitis vinifera
tcttgtggagaactccagcag

SEQ ID NO: 52 - DNA, 21 bp, Solanum lycopersicum
tatctatactcgttatcgcac

SEQ ID NO: 53 - DNA, 21 bp, Solanum lycopersicum
tatctatactcgtaatcgctc

SEQ ID NO: 54 - DNA, 21 bp, Solanum lycopersicum
tatctcatatggaattcgcgc

SEQ ID NO: 55 - DNA, 21 bp, Triticum aestivum
ttaacaggtgagtcgaatcag

SEQ ID NO: 56 - DNA, 21 bp, Triticum aestivum
ttaacaggtgagtcgaatcat

SEQ ID NO: 57 - DNA, 21 bp, Zea mays
tcaactctgagagtttcgcat

SEQ ID NO: 58 - DNA, 21 bp, Zea mays
ttaccatgacattaacgtcgc